(19) United States
(12) Patent Application Publication
(10) Pub. No.: US 2023/0040450 A1
(43) Pub. Date: Feb. 9, 2023

(54) Sterile Sealed Secured IV Catheter Dressing

(71) Applicant: One IV Solutions, LLC, Rye Beach, NH (US)

(72) Inventors: Robert Helm, Rye Beach, NH (US); Adam Helm, Alameda, CA (US); Michael Barenboym, Boston, MA (US); Douglas W. Lawrence, Framingham, MA (US)

(21) Appl. No.: 17/807,712

(22) Filed: Jun. 18, 2022

Related U.S. Application Data
(60) Provisional application No. 63/211,755, filed on Jun. 17, 2021.

(51) Int. Cl. A61M 25/02; A61M 25/01; A61M 39/18; A61M 39/12

(52) U.S. Cl. CPC A61M 25/02; A61M 25/0111; A61M 39/18; A61M 39/12; A61M 2025/0266

(57) ABSTRACT

Devices, systems, and methods used to dress a catheter device are disclosed. The devices and systems include a catheter dressing system including a body, a closure member, an adhesive plate, and an extension tubing having a catheter connector coupled to a distal end. The catheter dressing system is displaceable over the extension tubing and the extension tubing passes through the closure member. The catheter connector is disposable within a chamber of the body to prevent movement of the catheter connector. The closure member is actuated to form a circumferential seal around the extension tubing to prevent contamination of the catheter connector and to prevent axial movement of the extension tubing. The adhesive plate can be adhered to a patient's skin to form a circumferential seal around a catheter skin puncture site. The method includes sliding the catheter dressing system along the extension tubing.

### RELATED APPLICATIONS

This application claims priority to U.S. Provisional Application No. 63/211,755, filed on Jun. 17, 2021 and titled “Sterile Sealed Secured IV Catheter Dressing” which is hereby incorporated by reference in its entirety.

### TECHNICAL FIELD

The present invention relates to methods and devices for circumferentially sealing and securing medical devices in order to decrease complication and failure rates. Medical devices include, but are not limited to, vascular access catheters or other devices inserted into the human body.

### BRIEF DESCRIPTION OF THE DRAWINGS

The embodiments disclosed herein will become more fully apparent from the following description and appended claims, taken in conjunction with the accompanying drawings. These drawings depict only typical embodiments, which will be described with additional specificity and detail through use of the accompanying drawings in which:

FIG. 1 is a perspective view of an embodiment of a catheter dressing system.

FIG. 2 is an end view of a proximal end of the catheter dressing system of FIG. 1.

FIG. 3 is side cross-sectional view of a portion of the catheter dressing system of FIG. 1.

FIG. 4 is a perspective view of an embodiment of a closure lock member of the catheter dressing system of FIG. 1.

FIG. 5 is a perspective view of an embodiment of a catheter connector of the catheter dressing system of FIG. 1.

FIG. 6A is a perspective view of a portion of an embodiment of a portion of a closure member of the catheter dressing system of FIG. 1 in an open state.

FIG. 6B is a side cross-sectional view of the closure member of FIG. 6A.

FIG. 7A is a perspective view of the closure member in a closed or sealing state.

FIG. 7B is a side cross-sectional view of the closure member of FIG. 7A.

FIG. 8 is a perspective view of a portion of another embodiment of a catheter dressing system.

FIG. 9 is a side-cross sectional view of the catheter dressing system of FIG. 8.

FIG. 10 is a perspective view of another embodiment of a catheter dressing system.

FIG. 11A is a side cross-sectional view of the catheter dressing system of FIG. 10 with another embodiment of a closure member in an open state.

FIG. 11B is a side cross-sectional view of the catheter dressing system of FIG. 10 with the closure member in a closed or sealing state.

FIG. 12A is a side cross-sectional view of another embodiment of a catheter dressing system with another embodiment of a closure member in an open state.

FIG. 12B is a side cross-sectional view of the catheter dressing system of FIG. 12A with the closure member in a closed or sealing state.

FIG. 13A is a side cross-sectional view of another embodiment of a catheter dressing system with another embodiment of a closure member in an open state.

FIG. 13B is a side cross-sectional view of the catheter dressing system of FIG. 13A with the closure member in a closed or sealing state

### DETAILED DESCRIPTION

Peripheral, central and other (e.g., intra-osseous, umbilical, etc.) vascular access catheter insertion and care may be complex and highly variable processes involving gathering, setting up, and using multiple disparate pieces of equipment. This may lead to a highly variable and contaminated result. Deficiencies of vascular access catheter care may fall into two groups: (1) inadequate catheter protection, leading to internal and external contamination of the catheter, and (2) inadequate catheter securement and stabilization, leading to tissue injury and catheter damage/dislodgement.

In certain instances, vascular access technology and application technique may not allow simple, clinically reproducible, and durable sterile-sealing protection of the vascular access catheter. Because dressing technology may not seal and fully protect the catheter and its insertion site, catheter contamination commonly occurs. Past and existing attempts at placing and preserving a sterile catheter and catheter site using “aseptic no touch” technique have generally failed and have significantly contributed to the high rate of vascular catheter access failure (e.g., 35-50% of peripheral vascular catheters fail). Compounding the problem of contamination is that vascular catheter securement and stabilization technology may be inadequate in certain circumstances, leading to the need for a set of “compensatory” measures such as dedicated securement devices and extension “j-tubing”— measures that may increase clinical complexity, discomfort, and cost. The cost and complexity of these measures can be a strong deterrent to their use.

It is therefore highly desirable in certain instances to have a device that seals, stabilizes, and secures an inserted vascular access catheter—or any device inserted into the body—in a way that is simple to apply, effective in achieving intended therapeutic goals, and able to achieve this goal in a highly reproducible manner.

The catheter dressing systems disclosed can provide a fully sealed and secured vascular access device. The catheter dressing systems may be used to fully seal and secure any type of medical device that is inserted into the body of a patient. For example, the catheter dressing systems disclosed herein can be used to dress and secure straight peripheral intravenous (IV) catheter, an integrated peripheral IV catheter, a peripherally inserted central (PIC) catheter, a midline catheter, a central line catheter, an umbilical catheter, intra-aortic balloon pump (IABP), left ventricular assist device (LVAD), extracorporeal membrane oxygenation (ECMO), percutaneous endoscopic gastrostomy (PEG) feeding tubes, percutaneous nephrostomy device, epidural pain catheters, temporary pacemaker access devices, etc. The disclosed catheter dressing systems may be used across a broad spectrum/arena of patient care, from battlefield or EMS/ER front line, to ICU and floor care, to outpatient care facilities.

The disclosed catheter dressing systems disclosed may be configured to decrease the number of separate dressing material components and separate sterile and unsterile packaged materials that need to be gathered and assembled at the patient's bedside for catheter dressing placement over a newly inserted catheter device or other medical device inserted into a patient's body. The separately packaged and other non-packaged materials that may be gathered to establish a secured and covered catheter device when not using the disclosed catheter dressing system can include: (1) a sheet of adhesive dressing; (2) a dedicated securement device; (3) dedicated antimicrobial device; and (4) supplemental tape.

Using the disclosed catheter dressing system, the number of dressing material components and separate sterile and unsterile packaged materials that may be gathered and assembled at the patient's bedside may be reduced to 1 (one).

The disclosed embodiments of the catheter dressing system are configured to decrease the number of individual steps involved in dressing and securing an inserted catheter device. When not using the disclosed catheter dressing systems, dressing and securing the catheter device can involve 16 or more individual, often one-handed (as the other hand is needed to hold the newly inserted catheter in place), steps: A. Obtaining and arranging at the beside: (1) obtaining, arranging on a bedside surface, opening, and removing a sterile clear adhesive dressing; (2) obtaining, arranging on the bedside surface, opening, and removing a dedicated stabilization device; (3) obtaining, arranging on the bedside surface, opening, and removing a dedicated antimicrobial device; (4) obtaining, arranging on the bedside surface, opening, and removing a Luer-based extension tube, and (5) obtaining, arranging on the bedside surface adjunctive tape. B. Application of these gathered and arranged materials to the newly inserted catheter: (6) attaching a Luer-lock connector of the extension tubing to a catheter hub of the newly inserted catheter device; (7) placing the dedicated securement device beneath the extension tube Luer-lock connector and catheter hub by lifting them away from the skin surface; (8) peeling off an adhesive backing of the stabilization device; (9) adhering an adhesive of the stabilization device to the patient's skin adjacent a catheter skin insertion site; (10) activating a closure/securement mechanism of the stabilization device around the Luer-lock connector and catheter hub; (11) placing the anti-microbial device around the catheter-skin insertion site; (12) peeling off a central adhesive backing portion of a clear adhesive dressing; (13) placing the clear adhesive dressing over the connector hub and catheter device and adhering it to the surrounding skin surface; (14) peeling off a remaining adhesive backing from a rim/border portion of the adhesive dressing; (15) adhering the rim/border portion of the clear adhesive dressing to the skin; and (16) placing additional supplemental tape strips (e.g., strips included in an adhesive dressing kit or separate non-sterile strips from a roll).

When using the disclosed catheter dressing system, the number of individual steps may be decreased to 6 or less: (1) obtaining, opening and removing a catheter dressing system from its sterile packaging; (2) attaching a catheter connector of an extension tube to a hub of the inserted catheter device; (3) sliding the catheter dressing system over the extension tube into position over the catheter device and catheter connector; (4) closing a closure member of the catheter dressing system around the extension tube; (5) peeling off an adhesive backing of the an adhesive plate; and (6) adhering the adhesive plate to the skin.

Certain disclosed embodiments of the catheter dressing system have two states: open and closed. In the open state it can be easily placed over any catheter device without disturbing the catheter device. In the closed state, it provides two circumferential seals—one with the skin surrounding a catheter of the catheter device and its skin insertion site, and one around an extension tubing or other device attached to a hub of the catheter device. In the closed state, the catheter dressing system can seal the catheter and skin insertion site against external contamination and stabilize and secure the catheter device relative to the skin insertion site.

In one embodiment, the sterile sealing securing dressing is pre-mounted around a segment of tubing. A catheter device connector is attached to the distal end of this tubing and a needle-free or other connector is attached to the proximal end of the tubing. The open sterile sealing securing dressing is pre-mounted on the tubing between the two connector ends, the dressing having the ability to slide freely along the tubing through its two open circumferential orifices, both of which are in their open and non-sealed state. The pre-mounting of the dressing around the segment of tubing with its attached connectors allows several advantages: (1) the dressing and tubing connector can be packaged together as one sterile item in one sterile package that eliminates the need to open and combine multiple individual components, (2) the dressing can be packaged so that it is adjacent to the proximal connector (needle-free valve end away from the patient), leaving the distal tubing and connector easily available for finger-grasping and attachment to the catheter hub, (3) once the catheter connector is attached to the catheter hub and the dressing slid into final mounting position around the catheter hub-connector complex, the dressing can be grasped to stabilize the catheter while the two sterile seals are effected. The first circumferential sterile seal is achieved by removing the adhesive backing from the dressing and adhering the dressing to the skin, and the circumferential second is achieved by deploying the seal mechanism around the exiting tubing. The net result of this process is that two simple pre-integrated components—the dressing and the tubing with its attached mating connecter—can be treated as one simple unit, requiring only the opening of one package and the grasping of only this one simple unit. This single unit can then be used to fully sterilely seal and secure an inserted catheter in a way that is simple, intuitive, user-friendly, rapid, time and resource efficient, and highly reproducible.

Embodiments may be understood by reference to the drawings, wherein like parts are designated by like numerals throughout. It will be readily understood by one of ordinary skill in the art having the benefit of this disclosure that the components of the embodiments, as generally described and illustrated in the figures herein, could be arranged and designed in a wide variety of different configurations. Thus, the following more detailed description of various embodiments, as represented in the figures, is not intended to limit the scope of the disclosure, but is merely representative of various embodiments. While the various aspects of the embodiments are presented in drawings, the drawings are not necessarily drawn to scale unless specifically indicated.

Reference throughout this specification to “an embodiment” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

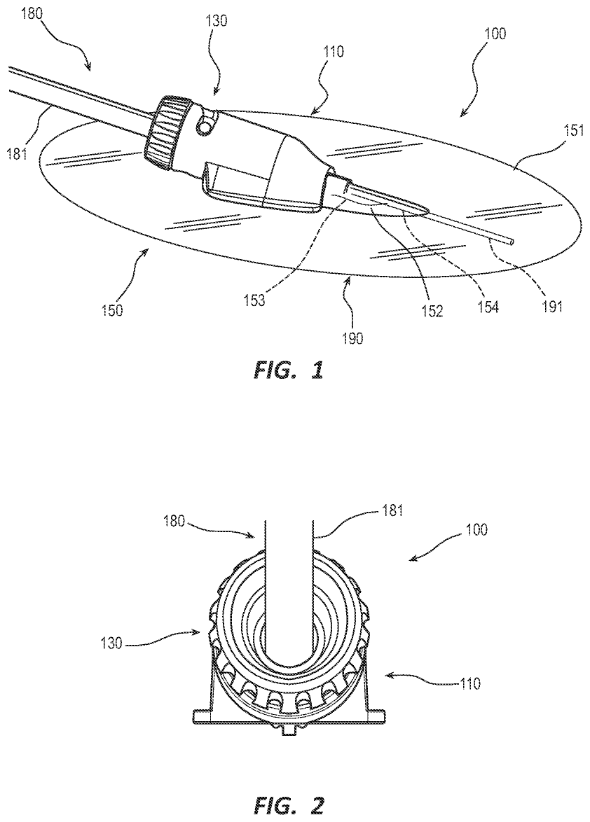

FIG. 1 illustrates an embodiment of a catheter dressing system 100. As illustrated, the catheter dressing system 100 can include a body 110, a closure member 130, an adhesive plate 150 coupled to the body 110, and an extension tube 180. The catheter dressing system 100 may seal a catheter device 190 against external contamination and secure the catheter device against movement relative to a skin puncture site by providing a first circumferential seal around a catheter 131 of a catheter device 190 at a skin puncture site and a second circumferential seal around a tubing 181 of the extension tube 180 when the closure member 130 is in a closed or sealing state, as shown in FIG. 2. FIG. 2 illustrates a proximal end of the catheter dressing system 100. As illustrated, the tubing 181 of the extension tube 180 passes through the closure member 130 and extends proximally from the body 110. In the closed state, the closure member 130 circumferentially seals around the tubing 181 to provide the second circumferential seal, as will be described below.

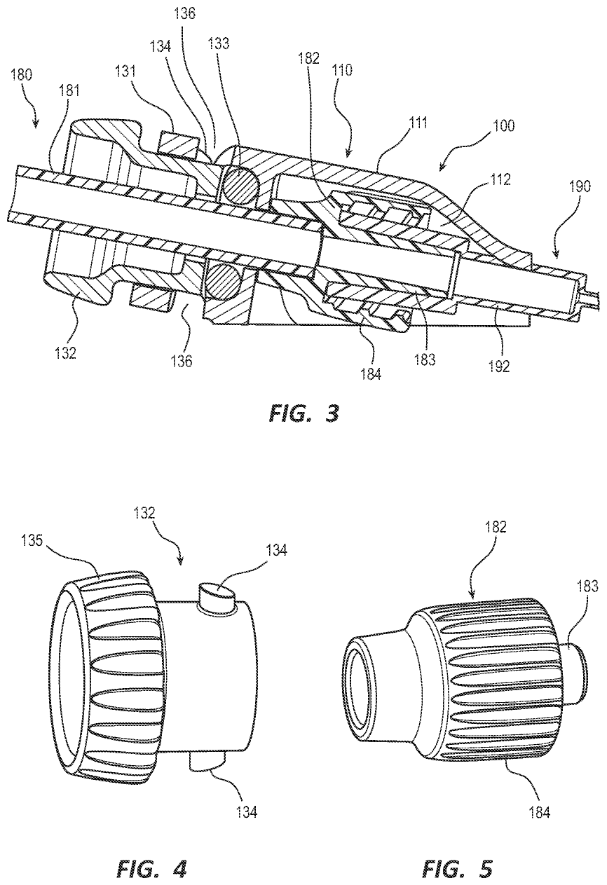

FIG. 3 illustrates a cross-section of the catheter dressing system 100 with the adhesive plate 150 removed. As illustrated, the body 110 comprises a shell 111 that defines a chamber 112. The shell 111 can be include any suitable rigid or semi-rigid material and be formed by any suitable technique, such as injection molding and thermoforming. A contour of the shell 111 may be shaped to reduce in size from a proximal portion to a distal portion. A contour of the shell chamber 112 may be shaped to substantially match an external shape of a catheter connector 182 of the extension tubing 180. When the catheter connector 182 is disposed within the shell chamber 112, the walls of the shell 111 secure the catheter connector 182 in place to prevent movement of the catheter connector 182 relative to the body 110. For example, a distal portion of the catheter connector 182 may engage with a distal wall of the shell 111, a proximal portion of the connector 182 may engage with a proximal wall of the shell 111, and a top portion of the connector 182 may engage with a top portion of the wall of the shell 111.

In some embodiments, the adhesive plate 150 may be coupled to the shell 111, as shown in FIG. 1. The adhesive plate 150 may include any suitable type material to form a flexible film, such as polyurethane. Other materials are within the scope of this disclosure. In some embodiments, the adhesive plate 150 may include a flexible film that is moisture vapor transmissible. The adhesive plate 150 can include a peripheral portion 151 and a dome portion 152. An adhesive can coat a lower surface of the peripheral portion 151. A release liner can cover the adhesive until the catheter dressing system 100 is ready to be applied to a patient. The adhesive may be configured to releasably attach the adhesive plate 150 to the patient and to form the first circumferential seal around the catheter 191 and the skin puncture site. The first circumferential seal can prevent microbes from migrating along the patient's skin to the skin puncture site, along the catheter 191, and into the patient's body. The dome portion 152 may define a plate chamber 153 that is communication with the shell chamber 112. A portion of the catheter device 190 and the skin puncture site can be disposed within the plate chamber 153. In some embodiments, the dome portion 152 may be clear to provide visualization of the catheter 191 and the skin puncture site. The adhesive plate 150 can include an aperture 154 sized to allow passage of the extension tubing 180 and the catheter device 190 when the catheter dressing system 100 is installed on the patient.

As illustrated in FIGS. 3 and 5, the extension tubing 180 includes a tube 181 made of a flexible material. The catheter connector 182 can be coupled to a distal end of the tube 181. The catheter connector 182 may be configured as a male-Luer lock fitting having a male-Luer slip portion 183 and a locking nut portion 184. Other types of fittings are within the scope of this disclosure. The catheter connector 182 may be coupled to a hub 192 of the catheter device 190. The hub 192 may include a fitting to form a fluid tight seal with the fitting of the catheter connector 182 (e.g., a female Luer-lock fitting). An outer surface of the catheter connector 182 can be contoured to securely fit or nest within the shell chamber 112 as previously described. In some embodiments, an adaptor (not shown), such as a female Luer-lock fitting, may be coupled to a proximal end of the tube 181 to allow infusion and/or withdrawal of fluids through the extension tube 180. In other embodiments, a needleless valve connector may be coupled to the proximal end of the tube 181.

As further illustrated in FIG. 3, the closure member 130 may be disposed proximally of the shell chamber 112. In the depicted embodiment, the closure member 130 is an integral part of the body 110. In other embodiments, the closure member 130 may be a separate component coupleable to the shell 111. The closure member 130 can include a receiving portion 131, a closure adaptor 132, and a gasket 133. The gasket 133 can be disposed within the receiving portion 131 against a proximal wall of the shell chamber 112. In the illustrated embodiment, the gasket 133 is an O-ring configured to circumferentially seal around the tube 181 when compressed by the closure adaptor 132 to provide the second circumferential seal. In other embodiments, the gasket 133 may be any suitable device capable of providing a circumferential seal on the tube 181 when activated. The closure adaptor 132 may be rotationally and longitudinally displaceable within the receiving portion 131. As shown in FIG. 4, the closure adaptor 132 may include one or more radial projections 134 configured to be disposed within one or more slots 136 of the receiving portion 131 to provide a bayonet-type of locking connection between the receiving portion 131 and the closure adaptor 131. The closure adaptor 132 can include a knob 135 configured to be gripped by a user to facilitate rotation of the closure adaptor 131.

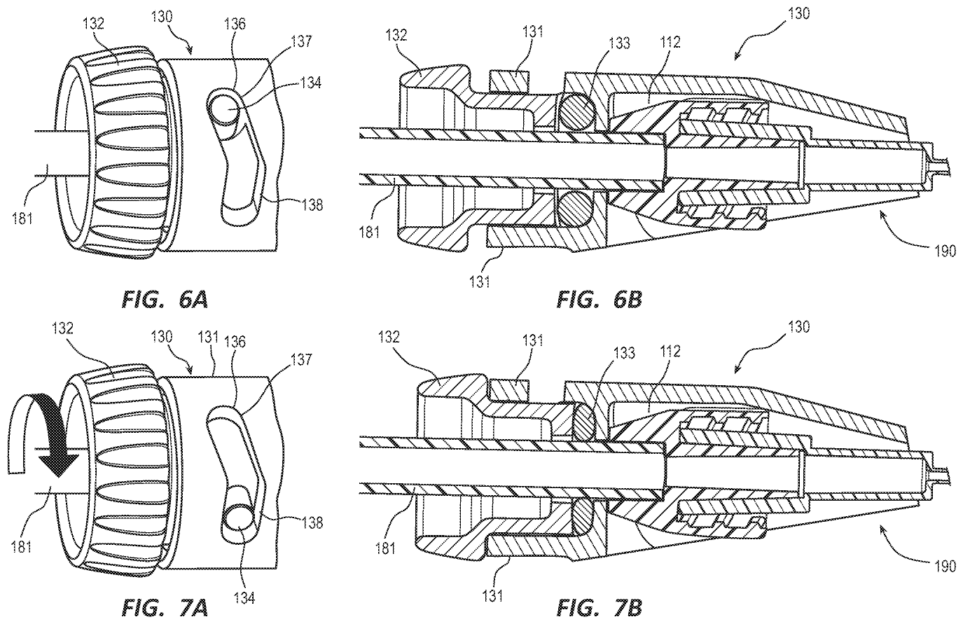

FIGS. 6A and 6B illustrate the closure member 130 in the open state. As illustrated, the tube 181 passes through the closure member 130 and the catheter dressing system 100 is free to be displaced along a length of the tube 181 because the gasket 133 is not sealing against the tube 181. The radial projection 134 is disposed within a first portion 137 of the slot 136 to position the closure adapter 132 in a proximal position. As shown, the first portion 137 may be angled distally. A second portion 138 can be substantially perpendicular relative to a longitudinal axis of the receiving portion 131.

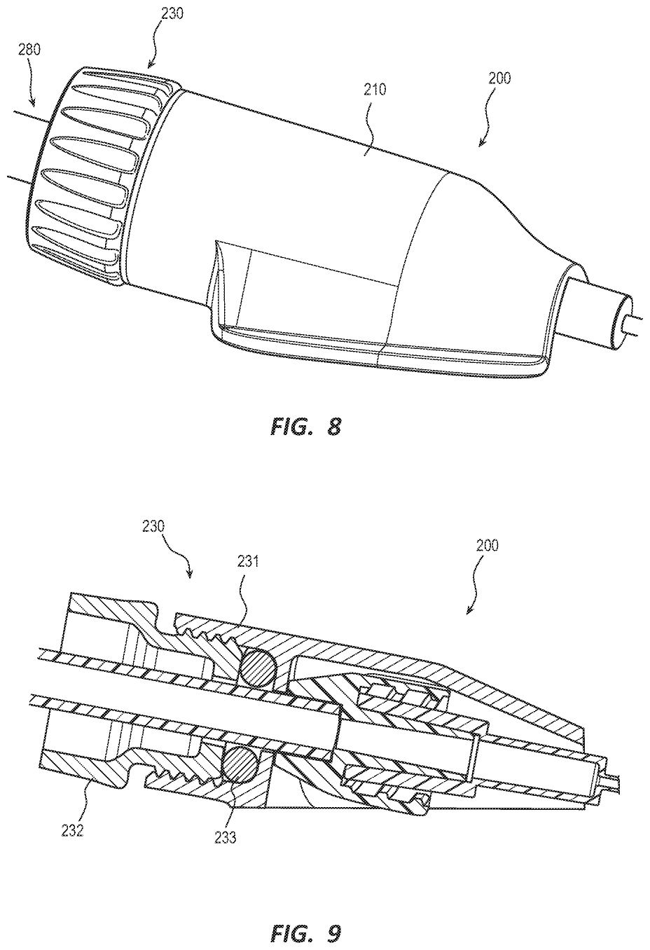

FIGS. 7A and 7B illustrate the closure member 130 in the closed or sealed state. As illustrated, the closure adaptor 132 may be rotated in a first direction, as indicated by the arrow, to cause the radial projection 134 to be displaced within the first portion 137 of the slot 136. As the radial projection 134 is displaced, the angle of the first portion causes the closure adaptor 132 to be displaced distally, as indicated by the arrow. When displaced distally, the closure adaptor 132 can compress the gasket 133 resulting in radial inward displacement of the material of the gasket 133 to form the second circumferential seal around the tube 181. In the closed state, the second circumferential seal can prevent microbes from migrating along the tube 181 and into the shell chamber 112 causing contamination of the catheter device 190. In the closed state, the second circumferential seal can secure the catheter device 190 in place to prevent movement of the catheter device 190 relative to the skin insertion site. With continued rotation of the closure adaptor 132 in the first direction, the radial projection 134 is disposed in the second portion 138 resulting in the closure adaptor 132 locked in a distal position to compress the gasket 133 and maintain the closure member 130 in the closed state. In some embodiments, the closure adaptor 132 may be rotated in a second direction, opposite the first direction, resulting in proximal displacement of the closure adaptor 132 and return of the closure member 130 to the open state.

FIGS. 8 and 9 depict an embodiment of a catheter dressing assembly 200 that resembles the catheter dressing assembly 100 described above in certain respects. Accordingly, like features are designated with like reference numerals, with the leading digit incremented to “2.” For example, the embodiment depicted in FIGS. 8 and 9 includes a closure member 230 that may, in some respects, resemble the closure member of FIG. 3. Relevant disclosure set forth above regarding similarly identified features thus may not be repeated hereafter. Moreover, specific features of the closure member 130 and related components shown in FIGS. 1-7B may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the catheter dressing system 200 and related components depicted in FIGS. 8 and 9. Any suitable combination of the features, and variations of the same, described with respect to the catheter dressing system 100 and related components illustrated in FIGS. 1-7B can be employed with the catheter dressing system 200 and related components of FIGS. 8 and 9, and vice versa. This pattern of disclosure applies equally to further embodiments depicted in subsequent figures and described hereafter, wherein the leading digits may be further incremented.

As illustrated in FIG. 8, the catheter dressing system 200 can include a body 210, a closure member 230, an adhesive plate (not shown), and an extension tubing 280. FIG. 9 depicts the closure member 230 may include an internally threaded receiving portion 231, and externally threaded closure adaptor 232, and a gasket 233. The external threads of the closure adaptor 232 can engage with the internal threads of the receiving portion 231 when the closure adaptor 232 is rotated in a first direction. When rotated in the first direction, the closure adaptor 232 may be distally displaced resulting in compression of the gasket 233 to form a second circumferential seal around a tube 281 of the extension tube 280.

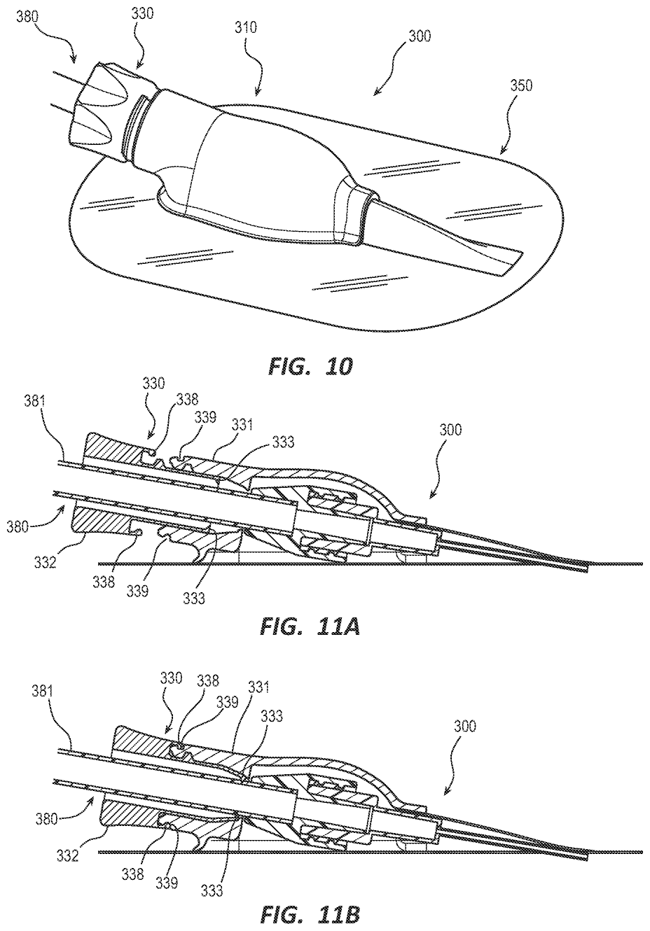

FIGS. 10, 11A, and 11B illustrates an embodiment of a catheter dressing system 300. As illustrated, the catheter dressing system 300 can include a body 310, a closure member 330, an adhesive plate 350, and an extension tube 380. FIG. 11A depicts the closure member 330 in an open state. The closure member 330 may include an internally threaded receiving portion 331, and externally threaded closure adaptor 332, and a gasket 333. The gasket 333 may be disposed on an internal wall at a distal end of the closure adaptor 332. The receiving portion 331 may include a distal portion that is tapered radially inward. The external threads of the closure adaptor 332 can engage with the internal threads of the receiving portion 331 when the closure adaptor 332 is rotated in a first direction. When rotated in the first direction, the closure adaptor 332 may be distally displaced resulting in radial inward displacement of the gasket 333, as shown in FIG. 11B, to transition the closure member 330 to a closed state. In the closed state, a second circumferential seal around a tube 381 of the extension tube 380 can be provided. In certain embodiments, the closure adaptor 332 may include a radial inwardly extending lip 338 and the receiving portion 331 may include a groove 339 configured to receive the lip 338 when the closure member 330 is in the closed state to maintain the closure member 330 in the closed state.

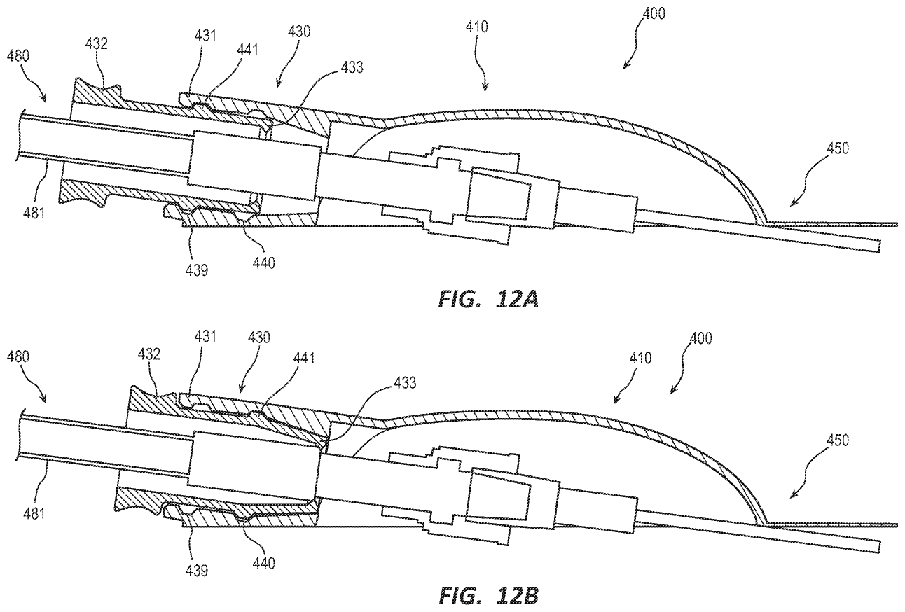

FIGS. 12A and 12B illustrate an embodiment of a catheter dressing system 400. As illustrated, the catheter dressing system 400 can include a body 410, a closure member 430, an adhesive plate 450, and an extension tube 480. FIG. 12A depicts the closure member 430 in an open state. The closure member 430 may include a receiving portion 431, a closure adaptor 432, and a gasket 433. The gasket 433 may be disposed on an internal wall at a distal end of the closure adaptor 432. The receiving portion 431 may include a distal portion that is tapered radially inward. The receiving portion 431 can include a proximal retention groove 439 and a distal retention groove 440. The closure adaptor 432 may include a retention ring 441 configured to engage with the proximal and distal retention grooves 439, 440. In the open state, the retention ring 441 is engaged with the proximal retention groove 439 to maintain the closure member 430 in the open state. FIG. 12B illustrates the closure member 430 in the closed state wherein the closure adaptor 432 may be longitudinally displaced distally to disengage the retention ring 441 from the proximal retention groove 439 and to radial inwardly displace the gasket 433, as shown in FIG. 11B, to transition the closure member 430 to a closed state. In the closed state, a second circumferential seal around a tube 481 of the extension tube 480 can be provided. In the closed state, the retention ring 441 can be engaged with the distal retention groove 440 to retain the closure member 430 in the closed state. In certain embodiments, the closure member 430 can be transitioned from the closed state to the open state when the closure member 430 is longitudinally displaced proximally.

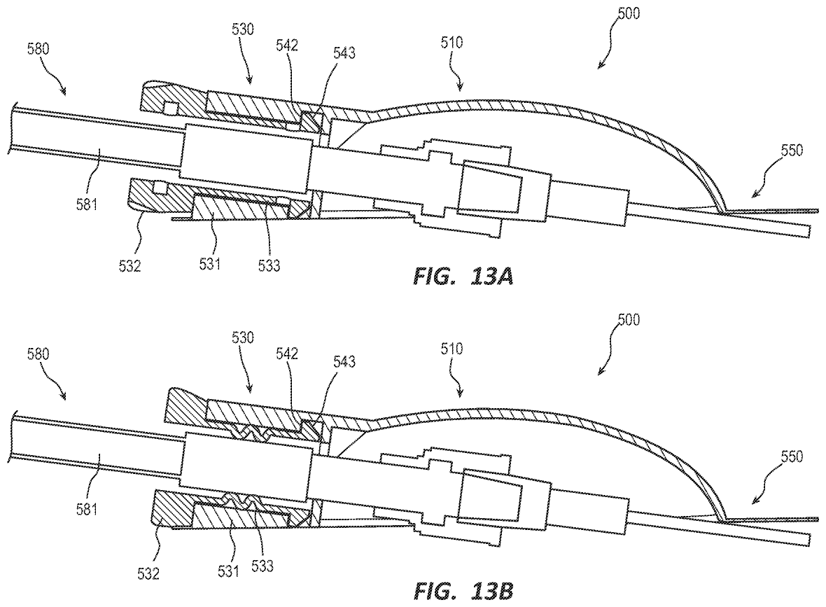

FIGS. 13A and 13B illustrate an embodiment of a catheter dressing system 500. As illustrated, the catheter dressing system 500 can include a body 510, a closure member 530, an adhesive plate 550, and an extension tube 580. FIG. 13A depicts the closure member 530 in an open state. The closure member 530 may include a receiving portion 531, a closure adaptor 532, and a gasket 533. The gasket 533 may be disposed proximally of a distal end of the closure adaptor 532. The receiving portion 531 may include a distal portion that is includes a shoulder 542 configured to engage with a catch 543 disposed at a distal end of the closure adaptor 532. In the open state, the catch 543 can be engaged with the shoulder 542 to prevent displacement of the closure adaptor 532 from the receiving portion 531. FIG. 13B illustrates the closure member 530 in the closed state wherein the closure adaptor 532 may be longitudinally displaced distally to longitudinally compress a portion of the closure adaptor 532 to provide the gasket 533 and to transition the closure member 530 to a closed state. In the closed state, a second circumferential seal around a tube 581 of the extension tube 580 can be provided. In certain embodiments, the closure member 530 can be transitioned from the closed state to the open state when the closure member 530 is longitudinally displaced proximally.

In use of any of the described embodiments of the catheter dressing systems 100, 200, 300, 400, 500, in order to decrease the number of steps required to achieve a fully sealed and secured catheter device, improve user-friendliness, and to reduce the variability in outcome, the catheter dressing system may be circumferentially mounted around a segment extension tube attached to a catheter connector configured to connect to a hub of a catheter device inserted into a patient's body. The catheter dressing system can be slid along the extension tube when the catheter dressing system is in an open state. The catheter dressing system can be moved to its final securing position around the inserted catheter device and catheter connector by sliding it down the extension tubing, and circumferential seals effected by: (1) removing an adhesive backing of an adhesive plate and adhering the adhesive plate to the skin to effect the first circumferential seal, and (2) actuating a closure member at an interface between the catheter dressing system and the segment of the extension tube that exits the catheter dressing system to effect the second circumferential seal when the catheter dressing system is in a closed state. In other words, the catheter dressing system can be configured to slide along the extension tube when in the open state, and to circumferentially seal and secure the catheter device when in an adherent and closed state.

In one embodiment, any of the catheter dressing systems 100, 200, 300, 400, 500 may be pre-mounted around a segment of an extension tube in an open state. A catheter connector can be coupled to a distal end of the extension tube and a needle-free or other connector can be attached to a proximal end of the extension tube. The catheter dressing system may be pre-mounted on the extension tube between the two connector ends, the catheter dressing system having the ability to slide freely along the extension tube through two open circumferential orifices, both of which are in open states. The pre-mounting of the catheter dressing system around the segment of tubing with its attached connectors may provide several advantages: (1) the catheter dressing system and a proximal connector can be packaged together as one sterile item in one sterile package that eliminates the need to open and combine multiple individual components; (2) the catheter dressing system can be packaged so that it is adjacent to the proximal connector (e.g., a needle-free valve end away from the patient), leaving a distal portion of an extension tube and a catheter connector easily available for finger-grasping and attachment to a catheter hub, (3) once the catheter connector is attached to the catheter hub and the catheter dressing system slid into final mounting position around the catheter device and catheter connector, the catheter dressing system can be grasped to stabilize the catheter device while two circumferential seals are effected. The first circumferential seal can be achieved by removal of an adhesive backing from the catheter dressing system and adhering the catheter dressing system to the skin, and the second circumferential can be achieved by deploying a closure member around the exiting extension tube. The result of this process can be that two pre-integrated components—the catheter dressing system and the extension tubing with its attached catheter connecter—can be treated as one unit, requiring only the opening of one package and the grasping of only this one unit. This single unit can then be used to sterilely seal and secure an inserted catheter device in a way that may be simple, intuitive, user-friendly, rapid, time and resource efficient, and highly reproducible.

The above delineated mounting sequence may eliminate a need to obtain, open, and arrange for sequential use, multiple individually packaged components. It also may eliminate a need to repeatedly reach for, grasp, and then sequentially use or attach these multiple individual components. Each individual component and its associated use methodology introduces room for error and for breaks in sterility, thereby increasing the potential suboptimal outcome. By eliminating the need for multiple components and their individual use steps, the disclosed catheter dressing system may decrease variability and improve outcomes. In addition, the embodiments disclosed may improve catheter stabilization and securement, and provide for durable sealing of the catheter device in order to fully protect the catheter device and its insertion site from the outside environment.

In the above description of embodiments, various features are sometimes grouped together in a single embodiment, figure, or description thereof for the purpose of streamlining the disclosure. This method of disclosure, however, is not to be interpreted as reflecting an intention that any claim requires more features than those expressly recited in that claim. Rather, as the following claims reflect, inventive aspects lie in a combination of fewer than all features of any single foregoing disclosed embodiment.

The phrases “coupled to” and “in communication with” refer to any form of interaction between two or more entities, including mechanical, electrical, magnetic, electromagnetic, fluid, and thermal interaction. Two components may be coupled to or in communication with each other even though they are not in direct contact with each other. For example, two components may be coupled to or in communication with each other through an intermediate component.

The directional terms “distal” and “proximal” are given their ordinary meaning in the art. That is, the distal end of a medical device means the end of the device furthest from the practitioner during use. The proximal end refers to the opposite end, or the end nearest to the practitioner during use.

“Fluid” is used in its broadest sense, to refer to any fluid, including both liquids and gases as well as solutions, compounds, suspensions, etc., which generally behave as fluids.

References to approximations are made throughout this specification, such as by use of the term “substantially.” For each such reference, it is to be understood that, in some embodiments, the value, feature, or characteristic may be specified without approximation. For example, where qualifiers such as “about” and “substantially” are used, these terms include within their scope the qualified words in the absence of their qualifiers. For example, where the term “substantially perpendicular” is recited with respect to a feature, it is understood that in further embodiments, the feature can have a precisely perpendicular configuration.

The terms “a” and “an” can be described as one, but not limited to one. For example, although the disclosure may recite a closure adaptor having “a radial projection,” the disclosure also contemplates that the closure adapter can have two or more radial projections.

Recitation in the claims of the term “first” with respect to a feature or element does not necessarily imply the existence of a second or additional such feature or element.

The claims following this written disclosure are hereby expressly incorporated into the present written disclosure, with each claim standing on its own as a separate embodiment. This disclosure includes all permutations of the independent claims with their dependent claims. Moreover, additional embodiments capable of derivation from the independent and dependent claims that follow are also expressly incorporated into the present written description.

Without further elaboration, it is believed that one skilled in the art can use the preceding description to utilize the invention to its fullest extent. The claims and embodiments disclosed herein are to be construed as merely illustrative and exemplary, and not a limitation of the scope of the present disclosure in any way. It will be apparent to those having ordinary skill in the art, with the aid of the present disclosure, that changes may be made to the details of the above-described embodiments without departing from the underlying principles of the disclosure herein. In other words, various modifications and improvements of the embodiments specifically disclosed in the description above are within the scope of the appended claims. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified. The scope of the invention is therefore defined by the following claims and their equivalents.

1. A catheter dressing system comprising:
a body comprising a shell defining a shell chamber;
a closure member coupled to the shell and in communication with the shell chamber, wherein the closure member is transitionable from an open state to a closed state;
an adhesive plate coupled to the body; and
a extension tubing comprising:
a tube disposed through the closure member; and
a catheter connector coupled to distal end of the tube, wherein the catheter connector is disposable within the shell chamber.

2. The catheter dressing system of claim 1,
wherein the adhesive plate is configured to form a first circumferential seal around a skin insertion site when the adhesive plate is adhered to a patient; and
wherein the closure member is configured to form a second circumferential seal around the tube of the extension tubing when in the closed state.

3. The catheter dressing system of claim 1, wherein the shell is configured to prevent movement of the catheter connector within the shell chamber.

4. The catheter dressing system of claim 1, wherein the closure member is configured to prevent movement of the extension tubing when in the closed state.

5. The catheter dressing system of claim 1, wherein the closure member comprises:
a receiving portion;
a closure adaptor configured to be displacably disposed within the receiving portion; and
a gasket,
wherein the gasket forms the second circumferential seal when the closure member is in the closed state.

6. The catheter dressing system of claim 5,
wherein the receiving portion comprises a slot comprising a first portion and a second portion;
wherein the closure adaptor comprises a radial projection configured to be disposed within the slot;
wherein the radial projection is disposed within the first portion when the closure member is in the open state; and
wherein the radial projection is disposed within the second portion when the closure member is in the closed state.

7. The catheter dressing system of claim 5,
wherein the closure adaptor is in a proximal position when the closure member is in the open state; and
wherein the closure adaptor is in a distal position when the closure member is in the closed state; and
wherein the gasket is longitudinally compressed by the closure adaptor when the closure member is in the closed state.

8. The catheter dressing system of claim 5,
wherein the receiving portion comprises an internal thread; and
wherein the closure adaptor comprises an external thread threadingly engageable with the internal thread of the receiving portion to displace the closure adaptor from a proximal position to a distal position.

9. The catheter dressing system of claim 5,
wherein the gasket is disposed at a distal end of the closure adaptor; and
wherein the gasket is radial inwardly displaced when the closure member is in the closed state.

10. The catheter dressing system of claim 1, wherein the catheter connector is configured to couple with a hub of a catheter device.

11. The catheter dressing system of claim 1, wherein the adhesive plate comprises:
a peripheral portion covered by an adhesive; and
a dome portion defining a dome chamber in communication with the shell chamber.

12. The catheter dressing kit, comprising:
a catheter dressing system comprising:
a body comprising a shell defining a shell chamber;
a closure member coupled to the shell and in communication with the shell chamber, wherein the closure member is transitionable from an open state to a closed state;
an adhesive plate coupled to the body; and
a extension tubing comprising:
a tube disposed through the closure member; and
a catheter connector coupled to distal end of the tube, wherein the catheter connector is disposable within the shell chamber; and
a needleless valve device couplable to a proximal end of the extension tubing.

13. A method of dressing a catheter device, comprising:
coupling a catheter connector coupled to an extension tubing to a hub of a catheter device;
sliding a catheter dressing system distally along the extension tubing wherein the catheter connector is disposed within a shell chamber of the catheter dressing system;
actuating a closure member of the catheter dressing system to form a first circumferential seal around the extension tubing;
removing an adhesive backing from an adhesive plate of the catheter dressing system; and
adhering the adhesive plate to a skin surface to form a second circumferential seal around a catheter skin insertion site.

14. The method of claim 13, wherein the number of components used is one.

15. The method of claim 13, wherein the second circumferential seal prevents contamination of an area surrounding the catheter skin insertion site.

16. The method of claim 13, wherein the first circumferential seal prevents contamination of the catheter connector and a catheter hub.

17. The method of claim 13, wherein the first circumferential seal prevents axial movement of the extension tubing.

18. The method of claim 13, wherein the catheter connector is restrained from movement within the shell chamber.
